(19) United States
(12) Patent Application Publication
(10) Pub. No.: US 2008/0214565 A1
(43) Pub. Date: Sep. 4, 2008

(54) Oxazolidinone Derivatives as Antimicrobials

(76) Inventors: Anita Mehta, Buffalo Grove, IL (US); Sonali Rudra, Delhi (IN); Ajjarapu Venkata Subrahmanya Rajo Rao, Haryana (IN); Ajay Singh Yadav, Ghaziabad (IN); Ashok Rattan, Delhi (IN)

Correspondence Address: RANBAXY INC., 600 COLLEGE ROAD EAST, SUITE 2100, PRINCETON, NJ, 08540, US

(21) Appl. No.: 10/552,322

(22) PCT Filed: Apr. 7, 2003
(86) PCT No.: PCT/IB03/01266

§ 371 Date: Jan. 19, 2008

(51) Int. Cl. A61K 31/496; C07D 413/10; A61P 31/04

(52) U.S. Cl. 514/254.02; 544/369

(57) ABSTRACT

The present invention relates to certain substituted phenyl oxazolidinones of formula I and H and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria such as multiply-resistant staphylococci, streptococci and enterococci as well as anaerobic organisms as Bacterioides spp. and Clostridia spp. species, and acid fast organisms such as Mycobacterium tuberculosis, Mycobacterium avium and Mycobacterium spp.

### FIELD OF THE INVENTION

The present invention relates to certain substituted phenyl oxazolidinones and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria such as multiply-resistant staphylococci, streptococci and enterococci as well as anaerobic organisms such as Bacterioides spp. and Clostridia spp. species, and acid fast organisms such as Mycobacterium tuberculosis, Mycobacterium avium and Mycobacterium spp.

### BACKGROUND OF THE INVENTION

Increasing antibacterial resistance in Gram positive bacteria has presented a formidable treatment problem. The enterococci, although traditionally non virulent pathogens, have been shown, when associated with Vancomycin resistance, to have an attributable mortality of approximately 40%. Staphylococcus aureus, the traditional pathogen of post operative wounds, has been resistant to Penicillin due to production of penicillinases. This resistance was overcome by the development of various penicillinase stable β lactams. But the pathogen responded by synthesizing a modified target penicillin binding protein-2′ leading to less affinity for β lactam antibiotics and a phenotype known as Methicillin Resistant S. aureus (MRSA). These strains, till recently were susceptible to Vancomycin, which inspite of its various drawbacks, has become the drug of choice for MRSA infections. Streptococcus pneumoniae is a major pathogen causing pneumonia, sinusitis and meningitis. Until very recently it was highly susceptible to penicillin. Recently though, different PBP 2′ strains with different susceptibility to penicillin have been reported from across the globe.

Oxazolidinones are a new class of synthetic antimicrobial agents which kill gram positive pathogens by inhibiting a very early stage of protein synthesis. Oxazolidinones inhibit the formation of ribosomal initiation complex involving 30S and 50S ribosomes leading to prevention of initiation complex formation. Due to their novel mechanism of action, these compounds are active against pathogens resistant to other clinically useful antibiotics.

WO 02/06278 application discloses phenyloxazolidinone derivatives as antimicrobials.

WO 93/23384 application discloses phenyloxazolidinones containing a substituted diazine moiety and their uses as antimicrobials.

WO 93/09103 application discloses substituted aryl and heteroaryl-phenyl-oxazolidinones useful as antibacterial agents.

WO 90/02744 application discloses 5-indolinyl-5β-amidomethyloxazolidinones, 3-(fused ring substituted) phenyl-5β-amidomethyloxazolidinones which are useful as antibacterial agents.

European Patent Publication 352,781 discloses phenyl and pyridyl substituted phenyl oxazolidinones.

European Patent Application 312,000 discloses phenylmethyl and pyridinylmethyl substituted phenyl oxazolidinones.

U.S. Pat. No. 5,254,577 discloses nitrogen heteroaromatic rings attached to phenyloxazolidinone.

U.S. Pat. Nos. 5,547,950 and 5,700,799 also disclose the phenyl piperazinyl oxazolidinones.

Chem. Pharm. Bull. 49(4) 347-352 (2001) describes conversion of 5-substituent oxazolidinone.

Chem. Pharm. Bull. 49(4) 353-360 (2001) describes 5-thiocarbonyl oxazolidinones.

Chem. Pharm. Bull. 49(4) 361-367 (2001) describes conversion of 5-thiocarbamate oxazolidinones.

WO 00/21960 describes heterocyclyl amino methyloxazolidinones as antibacterials.

Other references disclosing various phenyloxazolidinones include U.S. Pat. Nos. 4,801,600 and 4,921,869; Gregory W. A., et al., J. Med. Chem., 1989; 32: 1673-81; Gregory W. A., et al., J. Med. Chem., 1990; 33: 2569-78; Wang C., et al., Tetrahedron, 1989; 45: 1323-26; Brittelli, et al., J. Med. Chem., 1992; 35: 1156; Gordeev, Current Opinion in Drug Discovery & Development, 2001; Vol 4, No 4: 450-461; and Bio-organic and Medicinal Chemistry Letters, 1999; 9: 2679-2684; Antibacterial & Antifungal Drug Discovery & Development Summit, Strategic Research Institute, Jun. 28-29, 2001, Amsterdam, The Netherlands; Posters No. 1822, 1823, 1824, 1825, 1826, 1827, 1828, 1829, 1830, 1831, 1832, 1833, and 1834, 40^{th} Interscience Conference on Antimicrobial Agents and Chemotherapy, Sep. 17-20, 2000, Toronto, Canada; and Posters No 1023, 1040, 1041, 1042, 1043, 1044, 1045, 1046, 1047, 1048, 1049, 1050, and 1051, 41^{st} Interscience Conference on Antimicrobial Agents and Chemotherapy, Sep. 22-25, 2001, Chicago, USA.

### SUMMARY OF THE INVENTION

The invention involves the synthesis; identification and profiling of oxazolidinone molecules which have good activity against multiply resistant gram positive pathogens like MRSA, VRE and PRSP. Some of these molecules have activity against MDR-TB and MAI strains, while others have significant activity against important anaerobic bacteria.

The invention provides processes for the syntheses of phenyloxazolidinones derivatives which can exhibit significantly greater antibacterial activity against multiply resistant gram positive pathogens like MRSA, VRE and PRSP against MDR-TB and MAI strains, in order to provide safe and effective treatment of bacterial infections.

In accordance with one aspect of the invention, there are provided compounds having the structure of Formula I

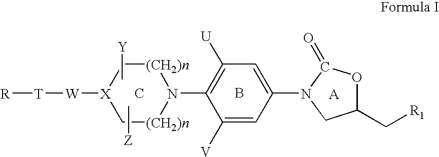

and their pharmaceutically acceptable salts, pharmaceutically acceptable solvates, esters, enantiomers, diastereomers, N-oxides, polymorphs, prodrugs or metabolites, wherein
T is a five to seven membered heterocyclic ring, substituted heterocyclic ring, aryl or substituted aryl, bound to the ring C with a linker W, for example preferred forms of T are aryl and five membered heteroaryl which are further substituted by a group represented by R, wherein R is H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is hydrogen, alkoxy, aryl, heteroaryl, amines, substituted amines, alkene substituted with aryl, heteroaryl or halogen; R₅ is H, C₁₋₁₂ alkyl, C₃₋₁₂ cycloalkyl, C₁₋₆ alkoxy, aryl, heteroaryl or C₁₋₆ alkyl substituted with one or more of F, Cl, Br, I or OH;
R₆ and R₇ are independently H, optionally substituted C₁₋₁₂ alkyl, C₃₋₁₂ cycloalkyl, C₁₋₆ alkoxy,
R₈ and R₉ are independently H, C₁₋₆ alkyl, F, Cl, Br, I, C₁₋₁₂ alkyl substituted with one or more of F, Cl, Br, I, OR₅, SR₄, or N(R₆,R₇);
R₁₀=H, optionally substituted C₁₋₁₂ alkyl, C₃₋₁₂ cycloalkyl, C₁₋₆ alkoxy, C₁₋₆ alkyl, aryl or heteroaryl;
n is an integer in the range from 0 to 3;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
Y and Z are independently hydrogen, C₁₋₆ alkyl, C₃₋₁₂ cycloalkyl, C₀₋₃ bridging groups;
U and V are independently hydrogen, optionally substituted C₁₋₆ alkyl, F, Cl, Br, I, C₁₋₁₂ alkyl substituted with one or more of F, Cl, Br, I;
W is CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂ or SO, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl; and
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaryl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy.

Particular compounds of Formula I have R₁ as ether linked isoxazole, amino-isoxazole, aminofuran, aminothiophene, or (un)substituted cinnamoyl and the most preferred compounds in this series would be prepared as the optically pure enantiomers having the (S)-configuration according to the Cahn-Ingold-Prelog notation at C₅ of the oxazolidinone ring.

In accordance with a second aspect of the invention, there are provided compounds of the Formula I containing D ring as furanyl, thienyl, and pyrrolyl ring systems and further substituted by substitutions G, J and L and are represented by Formula II

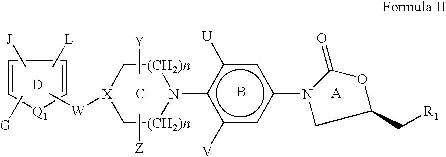

and their pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy,
U and V are independently hydrogen, optionally substituted C₁₋₆ alkyl, F, Cl, Br, I, C₁₋₁₂ alkyl substituted with one or more of F, Cl, Br, I;
Y and Z are independently hydrogen, C₁₋₆ alkyl, C₃₋₁₂ cycloalkyl, C₀₋₃ bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl carbonyl, C₁₋₆ alkylcarboxy, aryl or heteroaryl;
W is CH₂, C═O, CH₂NH, NHCH₂, CH₂NHCH₂, CH₂N(R₁₁)CH₂, CH₂N(R₁₁), CH(R₁₁), S, CH₂(C═O), NH, O, (CO)CH₂, N(R₁₁)CON(R₁₁), SO₂, SO, NR₁₁, N(R₁₁)C(═S)N(R₁₁); wherein R₁₁ is hydrogen, optionally substituted C₁₋₁₂ alkyl, C₃₋₁₂ cycloalkyl, C₁₋₆ alkoxy, C₁₋₄ alkyl carbonyl, C₁₋₆ alkylcarboxy, aryl or heteroaryl;
n is an integer in the range from 0 to 3;
Q₁ is O, S or NR₁₁, wherein R₁₁ is as defined above;
G, J, L are independently H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is as defined above; R₅ is H, C₁₋₁₂ alkyl, C₃₋₁₂ cycloalkyl, C₁₋₆ alkoxy, aryl or heteroaryl; C₁₋₆ alkyl substituted with one or more of F, Cl, Br, I or OH;
R₆ and R₇ are independently H, optionally substituted C₁₋₁₂ alkyl, C₃₋₁₂ cycloalkyl or C₁₋₆ alkoxy;
R₈ and R₉ are independently H, C₁₋₆ alkyl, F, Cl, Br, I, C₁₋₁₂ alkyl substituted with one or more of F, Cl, Br, I, OR₅, SR₄, N(R₆,R₇); and
R₁₀=H, optionally substituted C₁₋₁₂ alkyl, C₃₋₁₂ cycloalkyl, C₁₋₆ alkoxy, C₁₋₆ alkyl, aryl or heteroaryl.

In some compounds represented by Formula II, ring C may be 6-8 membered in size and the ring may have either two or three carbon atoms between each nitrogen atom, for example:

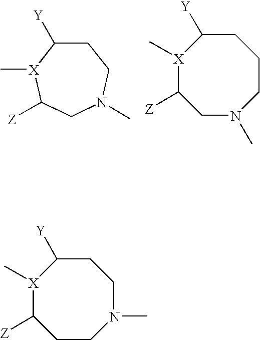

The ring C may be bridged to form a bicyclic system as shown below:

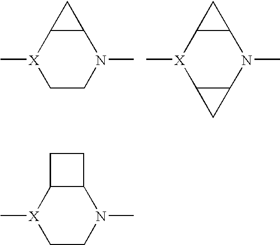

When ring C is optionally substituted at positions Y and Z with alkyl groups, cycloalkyl groups, fluoro group, carboxylic and corresponding esters, amides, substituted alkyls or bridging alkyl groups are as shown below:

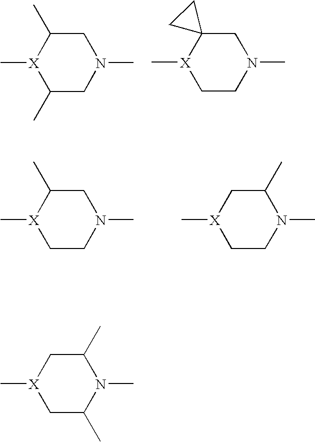

When ring C is 6 membered in size and X is —CH—(NR₁₁), or >CCH₂NR₁₁—, the following rings are preferred ones wherein R₁₁ is as defined earlier.

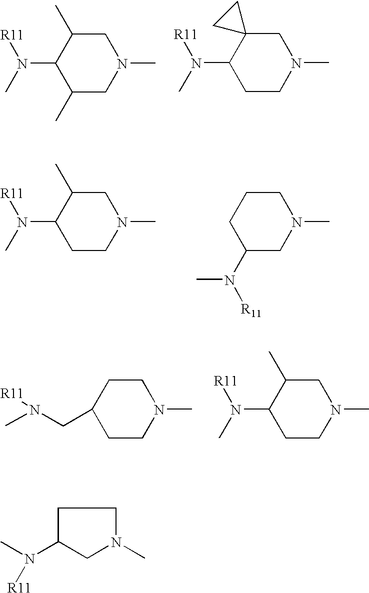

In addition to the above, ring C also includes the following structures:

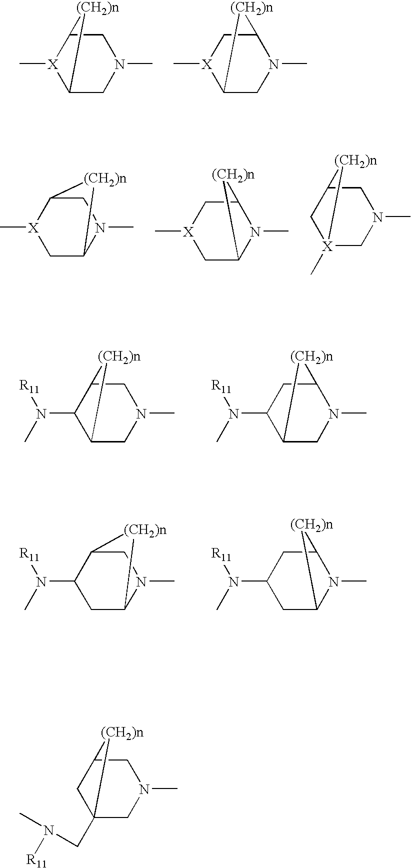

In accordance with a third aspect of the invention, there are provided compounds represented by Formula III

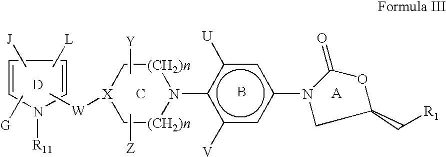

and their pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl, heteroaryl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy;
U and V are independently hydrogen, optionally substituted C₁₋₆ alkyl, F, Cl, Br, I, C₁₋₁₂ alkyl substituted with one or more of F, Cl, Br, I;
Y and Z are independently hydrogen, C₁₋₆ alkyl, C₃₋₁₂ cycloalkyl, C₀₋₃ bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
W is independently CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(—R₁₁), N(R₁₁)C(═S)N(R₁₁), SO₂ or SO, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
n is an integer in the range from 0 to 3;
G, J, L are independently H, C₁₋₆ alkyl % F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein 4 is as defined above and R₅ is H, C₁₋₁₂ alkyl, C₃₋₁₂ cycloalkyl, C₁₋₆ alkoxy, C₁₋₆ alkyl substituted with one or more of F, Cl, Br, I or OH, aryl or heteroaryl; R₆ and R₇ are independently H, optionally substituted C₁₋₂ alkyl, C₃₋₁₂ cycloalkyl, C₁₋₆ alkoxy; R₈ and R₉ are independently H, C₁₋₆ alkyl, F, Cl, Br, I, C₁₋₁₂ alkyl substituted with one or more of F, Cl, Br, I, OR₅, SR₄, N(R₆,R₇); and R₁₀=H, optionally substituted C₁₋₁₂ alkyl, C₃₋₁₂ cycloalkyl, C₁₋₆ alkoxy, C₁₋₆ alkyl, aryl or heteroaryl.

In accordance with a fourth aspect of the invention, there are provided compounds represented by Formula IV

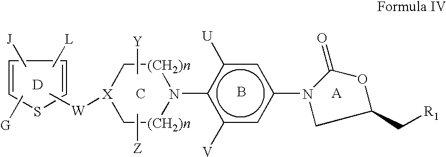

and their pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl, heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy;
U and V are independently hydrogen, optionally substituted C₁₋₆ alkyl, F, Cl, Br, I, C₁₋₁₂ alkyl substituted with one or more F, Cl, Br, I;
Y and Z are independently hydrogen, C₁₋₆ alkyl, C₃₋₁₂ cycloalkyl, C₀₋₃ bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
W is independently CH₂, CO, CH₂NH, —NHCH₂, —CH₂NHCH₂, —CH₂—N(R₁₁)CH—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂ or SO, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
n is an integer in the range from 0 to 3;
G, J, L are independently H, C₁₋₆ alkyl, F, Cl, Br, I, —CN, COR₅, COOR₅, N(R₆, R₇), NHCOC(R₈, R₉), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is as defined above; R₅ is H, C₁₋₁₂ alkyl, C₃₋₁₂ cycloalkyl, C₁₋₆ alkoxy, C₁₋₆ alkyl substituted with one or more of F, Cl, Br, I or OH, aryl or heteroaryl; R₆ and R₇ are independently H, optionally substituted C₁₋₁₂ alkyl, C₃₋₁₂ cycloalkyl, C₁₋₆ alkoxy; R₈ and R₉ are independently H, C₁₋₆ alkyl, F, Cl, Br, I, C₁₋₁₂ alkyl substituted with one or more of F, Cl, Br, I, OR₅, SR₄, N(R₆,R₇); R₁₀=H, optionally substituted C₁₋₁₂ alkyl, C₃₋₁₂ cycloalkyl, C₁₋₆ alkoxy, C₁₋₆ alkyl, aryl or heteroaryl.

A particular compound of Formula IV is as follows:

### Compound No. 12:

### (S)—N-[1-[[3-[3-Fluoro-4-[N-1-[4-{2-thienyl-(5-nitro)methyl}]piperazinyl]phenyl]-2-oxo-5-oxazolidinyl]methyl]]-thiourea

In accordance with a fifth aspect of the invention, there are provided compounds represented by Formula V

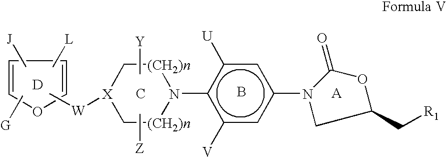

and their pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl, heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy; preferably R₂, R₃, R₄ are (un)substituted cinnamoyl and isoxazolyl ring;
U and V are independently hydrogen, optionally substituted C₁₋₆ alkyl, F, Cl, Br, C₁₋₁₂ alkyl substituted with one or more of F, Cl, Br, I;
Y and Z are independently hydrogen, C₁₋₆ alkyl, C₃₋₁₂ cycloalkyl, C₀₋₃ bridging group;
X is H, CH, CH—S, CH—O, N, CHNR₁₁ or CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₄ alkylcarbonyl, C₁₋₆ alkylcarboxy, aryl or heteroaryl;
W is independently CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂, SO, wherein R₁₁ is hydrogen, optionally substituted C₁₋₁₂ alkyl, C₃₋₁₂ cycloalkyl, C₁₋₄ alkoxy, C₁₋₆ alkyl, C₁₋₆ alkylcarbonyl, C₁₋₆ alkylcarboxy, aryl or heteroaryl;
n is an integer in the range from 0 to 3;
G, J, L are independently H, C₁₋₆ alkyl, F, Cl, Br, I, —CN, COR₅, COOR₅, N(R₆, R₇), NHCOC(R₈, R₉), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₁₂ alkyl substituted with one or more of F, Cl, Br, I, OR₄, SR₄; wherein R₅ is H, C₁₋₁₂ alkyl, C₃₋₁₂ cycloalkyl, C₁₋₆ alkoxy, C₁₋₆ alkyl substituted with one or more of F, Cl, Br, I or OH, aryl or heteroaryl; R₆ and R₇ are independently H, optionally substituted C₁₋₁₂ alkyl, C₃₋₁₂ cycloalkyl, Clot alkoxy; R₈ and R₉ are independently H, C₁₋₆ alkyl, F, Cl, Br, I, C₁₋₁₂ alkyl substituted with one or more of F, Cl, Br, I, OR₅, SR₄, N(R₆,R₇); and R₁₀=H, optionally substituted C₁₋₁₂ alkyl, C₃₋₁₂ cycloalkyl, C₁₋₆ alkoxy, C₁₋₆ alkyl, aryl or heteroaryl.

A particular compound of Formula V is as follows:

### Compound No. 10

### (S)—N-[1-[[3-[3-fluoro-4-[N-1-[4-{2-furyl-(5-nitro)methyl}]piperazinyl]phenyl]-2-oxo-5-oxazolidinyl]methyl]]-thiourea

Compounds of the present invention can be useful antimicrobial agents, effective against a number of human and veterinary pathogens, particularly aerobic and Gram-positive bacteria, including multiply-antibiotic resistant staphylococci and streptococci, as well as anaerobic organisms such as Mycobacterium tuberculosis and other mycobacterium species.

For preparing pharmaceutical compositions from the compounds described by this invention, inert, pharmaceutically acceptable carriers can be either solid or liquid. Solid form preparations include powders, tablets, dispersible granules, capsules, cachets, suppositories and ointments. A solid carrier can be one or more substances which may also act as diluents, flavouring agents, solubilizers, lubricants, suspending agents, binders, or tablets disintegrating agents; it can also be as finely divided solid which is in admixture with the finely divided active compound. For the preparation of tablets, the active compound is mixed with carrier having the necessary binding properties in suitable proportions and compacted in the shape and size desired. The powders and tablets preferably contain from about 5 to about 70 percent of the active ingredient. Suitable solid carriers are lactose, pectin, dextrin, starch, gelatin, tragacanth, low melting wax, cocoa butter and the like. The term “preparation” is intended to include the formulation of the active compound with encapsulating material as carrier providing a capsule in which the active component (with or without other carriers) is surrounded by carrier, which is thus in association with it. Similarly, capsules can be used as solid dosage forms suitable for oral administration.

Liquid form preparations include solutions suspensions and emulsions. As an example may be mentioned water or water-propylene glycol solutions for parenteral injection. Such solutions are prepared so as to be acceptable to biological systems (isotonicity, pH, etc.). Liquid preparations can also be formulated in solution in aqueous polyethylene glycol solution. Aqueous solutions suitable for oral use can be prepared by dissolving the active component in water and adding suitable colorants, flavours, stabilizing, and thickening agents as desired. Aqueous suspension suitable for oral use can be made by dispersing the finely divided active component in water with viscous material, i.e., natural or synthetic gums, resins, methyl cellulose, sodium carboxymethyl cellulose and other well-known suspending agents.

Ointment preparations contain heavy metal salts of a compound of Formula I with a physiologically acceptable carrier. The carrier is desirably a conventional water-dispersible hydrophilic or oil-in-water carrier, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

The pharmaceutical preparation can be in unit dosage form. In such forms, the preparation is subdivided into unit doses containing appropriate quantities of the active component. The unit dosage form can be a packaged preparation, the package containing discrete capsules, powders in vials or ampoules and ointments capsule, cachet, tablet, gel, or cream itself or it can be the appropriate number of any of these packaged forms.

The quantity of active compound in a unit dose of preparation may be varied or adjusted from less than 1 mg to several grams according to the particular application and the potency of the active ingredient.

In therapeutic use as agents for treating bacterial infections the compounds utilized in the pharmaceutical method of this invention are administered at the initial dosage of about 3 mg to about 40 mg per kilogram daily. The dosages, however, may be varied depending upon the requirements of the patient and the compound being employed. Determination of the proper dosage for a particular situation is within the smaller dosages which are less than the optimum dose. Small increments until the optimum effect under the daily dosage may be divided and administered in portions during the day if desired.

In one aspect, the invention provides processes for the synthesis of compounds of Formulae I, II, III, IV and V. Pharmaceutically acceptable non-toxic acid addition salts of the compounds of the present invention of Formulae I, II, IV, I and V may be formed with inorganic or organic acids, by methods well known in the art.

The present invention also includes within its scope prodrugs of the compounds of Formulae I, III, IV, I and V. In general, such prodrugs will be functional derivatives of these compounds which readily get converted in vivo into defined compounds. Conventional procedures for the selection and preparation of suitable prodrugs are known to the artisan of ordinary skill in the art.

The invention also includes pharmaceutically acceptable salts, pharmaceutically acceptable solvates, the enantiomers, diastereomers, N-oxides, prodrugs, metabolites in combination with a pharmaceutically acceptable carrier and optionally included excipients.

Other advantages of the invention will be set forth in the description which follows, and in part will be apparent from the description, or may be learned by the practice of the invention.

### DETAILED DESCRIPTION OF THE INVENTION

The compounds described herein represented by general Formula I may be prepared by the reaction sequence as shown in Scheme I:

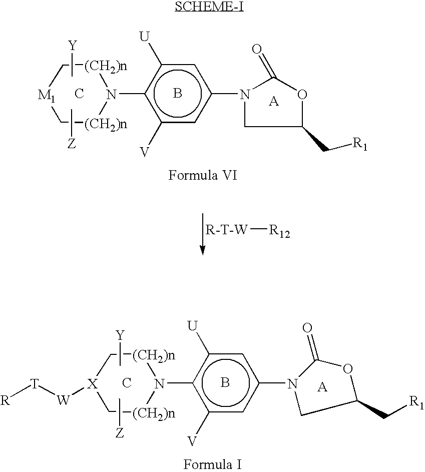

In Scheme I, the amine of structure of Formula VI wherein

M₁ is NH, NHR₁₃, —CH₂NHR₁₃, wherein R₁₃ is H, ethyl, methyl, isopropyl, acetyl, cyclopropyl, alkoxy;
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy,
U and V are independently hydrogen, optionally substituted C₁₋₆ alkyl, F, Cl, Br, I, C₁₋₁₂ alkyl substituted with one or more of F, Cl, Br, I;
Y and Z are independently hydrogen, C₁₋₆ alkyl, C₃₋₁₂ cycloalkyl, C₀₋₃ bridging group;
is reacted with a heteroaromatic compound of Formula R-T-W—R₁₂ wherein
T is a five to seven membered heterocyclic ring, substituted heterocyclic ring, aryl or substituted aryl, bound to the ring C with a linker W, for example preferred forms of T are selected from aryl and five membered heteroaryl which are further substituted by a group represented by R, wherein R is H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is hydrogen, alkoxy, aryl, heteroaryl, amines, substituted amines, alkene substituted with aryl, heteroaryl or halogen; R₅ is H, C₁₋₁₂ alkyl, C₃₋₁₂ cycloalkyl, C₁₋₆ alkoxy, aryl, heteroaryl or C₁₋₆ alkyl substituted with one or more of F, Cl, Br, I or OH;
W is CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂ or SO, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl; and
R₁₂ is a suitable leaving group well known to one of ordinary skill in the art such as fluoro, chloro, bromo, SCH₃, —SO₂CH₃, —SO₂CF₃, Tos or OC₆H₅, —COOH or —CHO, etc.

For the preparation of compounds of Formula I (wherein W is equal to CH₂), the corresponding aldehyde can be used through a process of reductive amination and is attached to amine of Formula VI.

Similarly, for the preparation of compound of Formula I wherein W is equal to C═O, the corresponding acid can be used and the amino compound of Formula VI can be acylated through activated esters in the presence of condensing agents, such as 1,3-dicyclohexylcarbodiimide (DCC) and 1-(3-dimethylaminopropyl)-3-ethylcarbodiimide hydrochloride (EDC). Other methods of acylation can also be employed.

The preparation of the compound of Formula II can be accomplished as shown in Scheme II:

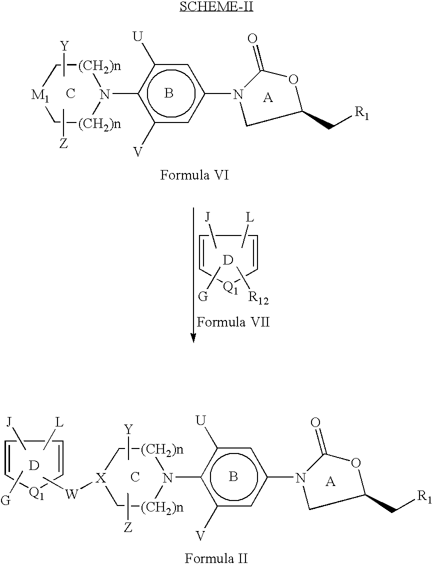

The reductive alkylation of the amine intermediate of Formula VI, wherein

M₁ is NH, NHR₁₃, —CH₂NHR₁₃, wherein R₁₃ is H, ethyl, methyl, isopropyl, acetyl, cyclopropyl, alkoxy;
R₁ is NHC(—O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy;
U and V are independently selected from hydrogen, optionally substituted C₁₋₆ alkyl, F, Cl, Br, I, C₁₋₁₂ alkyl substituted with one or more of F, Cl, Br, I; preferably U and V are hydrogen and fluoro;
Y and Z are independently hydrogen, C₁₋₆ alkyl, C₃₋₁₂ cycloalkyl, C₀₋₃ bridging group;
with the corresponding heterocyclic aldehydes of the Formula VII, wherein
Q₁ is O, S or NR₁₁, wherein R₁₁ is as defined above;
G, J, L are independently H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is as defined above; R₅ is H, C₁₋₁₂ alkyl, C₃₋₁₂ cycloalkyl, C₁₋₆ alkoxy, aryl or heteroaryl; C₁₋₆ alkyl substituted with one or more of F, Cl, Br, I or OH;
R₁₂ is a suitable leaving group well known to one of ordinary skill in the art such as fluoro, chloro, bromo, SCH₃, —SO₂CH₃, —SO₂CF₃, Tos or OC₆H₅, —COOH or —CHO, for example furaldehyde (Formula VII, wherein Q₁=O, G, J, L=H; R₁₂ is CHO), using known reducing agents well known to one of ordinary skill in the art such as sodium triacetoxyborohydride or sodium cyanoborohydride gave the products of Formula II (wherein W═CH₂) as shown in the Scheme II.

Alternatively, the compounds having carbonyl link can also be made by reacting heteroaromatic compound of the Formula VII, such as N-methylpyrrole with the amino compound of Formula VI in the presence of triphosgene or phosgene. The carbonyl linkers may also be introduced between heteroaromatic compound, such as 3-bromothiophene and the amine of Formula VI with carbon monoxide in the presence of a catalyst, such as bis(triphenylphosphine)palladium(II)chloride (Pd(PPh₃)₂Cl₂. The extended chain pyrroles having dicarbonyl linkers can also be obtained from treatment with oxalyl chloride and the amine of the Formula VI.

The reduction of the carbonyl linkers using the standard reducing agents results in the formation of methylene linkers.

The heteroaromatic compound of Formula VIE is reacted with the amino compound of Formula VI in the presence of ligands, such as tris(dibenzylideneacetone)dipalladium (Pd₂(dba)₃) and palladium diacetate (Pd(OAc)₂).

The reaction of compound of Formula VI with a compound of Formula VII can be carried out in a suitable solvent such as dimethylformamide, dimethylacetamide, acetonitrile, dimethylsulfoxide and ethylene glycol.

The reaction of compound of Formula VI with a compound of Formula VII is carried out in the presence of a suitable base, such as triethylamine, diisopropylethylamine, potassium carbonate, sodium carbonate and dipotassium hydrogenphosphate.

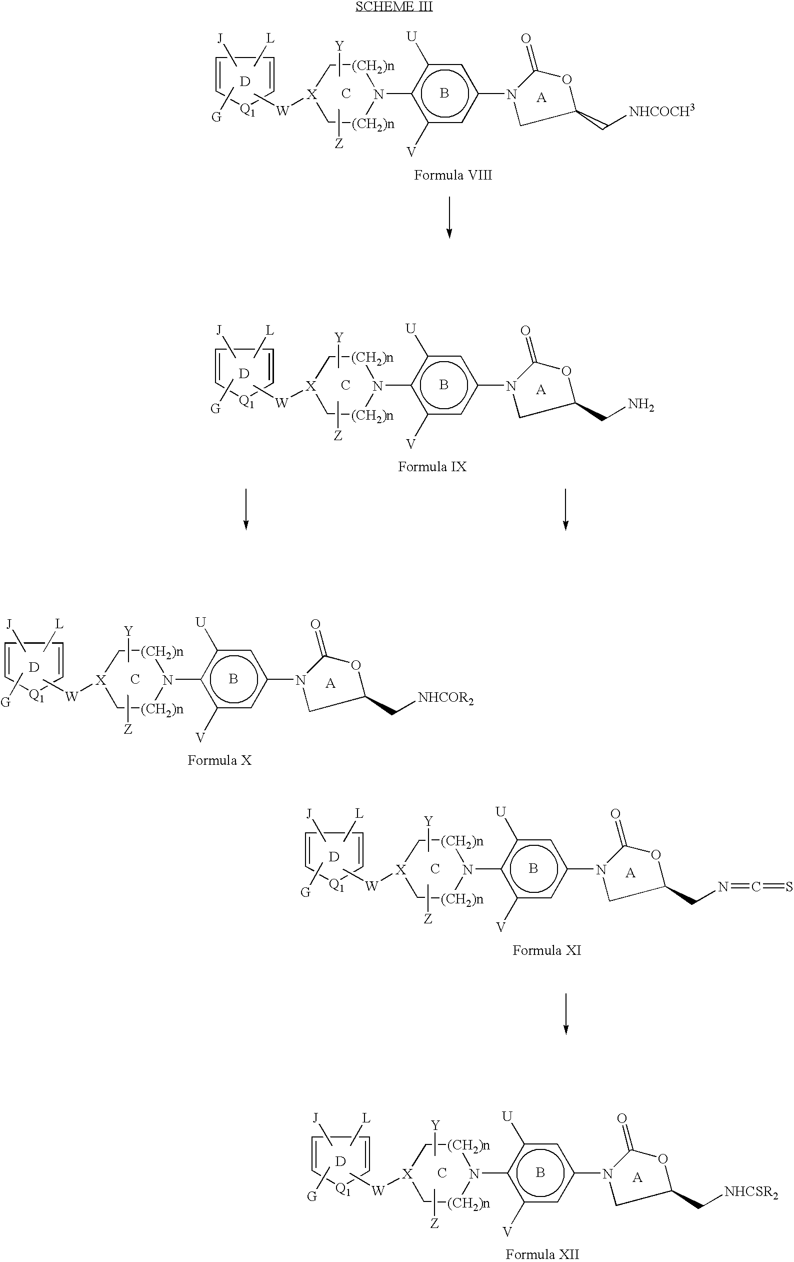

The compounds of Formula VIII (prepared as described in the patent application WO 02/06278) were used as starting materials for derivatisation as represented by Scheme III, wherein

U and V are independently hydrogen, optionally substituted C₁₋₆ alkyl, F, Cl, Br, I, C₁₋₁₂ alkyl substituted with one or more of F, Cl, Br, I;
Y and Z are independently hydrogen, C₁₋₆ alkyl, C₃₋₁₂ cycloalkyl, CO-3 bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₄ alkyl carbonyl, C₁₋₆ alkylcarboxy, aryl or heteroaryl;
W is CH₂, C═O, CH₂NH, NHCH₂, CH₂NHCH₂, CH₂N(R₁₁)CH₂, CH₂N(R₁₁), CH(R₁₁), S, CH₂(C═O), NH, O, (CO)CH₂, N(R₁₁)CON(R₁₁), SO₂, SO, NR₁₁, N(R₁₁)C(═S)N(R₁₁); wherein R₁₁ is hydrogen, optionally substituted C₁₋₁₂ alkyl, C₃₋₁₂ cycloalkyl, C₁₋₆ alkoxy, C₁₋₆ alkyl carbonyl, C₁₋₆ alkylcarboxy, aryl or heteroaryl;
n is an integer in the range from 0 to 3;
Q₁ is O, S or NR₁₁, wherein R₁₁ is as defined above;
G, J, L are independently H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is as defined above; R₅ is H, C₁₋₁₂ alkyl, C₃₋₁₂ cycloalkyl, C₁₋₆ alkoxy, aryl or heteroaryl; C₁₋₆ alkyl substituted with one or more of F, Cl, Br, I or OH;

The acetamide of Formula VIII is hydrolyzed with 1N hydrochloric acid to give the corresponding amine of Formula IX which is reacted with aryl carboxylic acids, such as Ar—COOH where Ar is (un) substituted cinnamic acids and heteroaryl carboxylic acids of Formula VII where R₁₂=COOH, is converted into the amide of Formula X. The acylation is carried out in the presence of condensing agents, such as 1,3-dicyclohexylcarbodiimide (DCC) and 1-(3-dimethylaminopropyl)-3-ethylcarbodiimide hydrochloride (EDC), along with 1-hydroxy benzotriazole (HOBT). Other methods of acylation can also be employed.

The acylation of the intermediate amine of Formula IX with heterocyclic acid of Formula VII, such as 2-furoic acid (Q₁=O; G, J, L=H; R₁₂=COOH) or aryl carboxylic acid, Ar—COOH where Ar=(un) substituted cinnamic acids gives products of Formula X.

Alternatively, the amine of Formula IX can be converted to the corresponding isothiocyanates of Formula XI with carbondisulfide and ethylchloroformate in the presence of a base and in a suitable solvent. The isocyanates can be further converted to thioureas of Formula XII on reaction with (un) substituted amine in the presence of a base.

The isocyanates of Formula XI is reacted with a (un)substituted amine to get compounds of Formula II. The reaction can be carried out in a suitable solvent, such as dimethylformamide, dimethylacetamide, dichloromethane or tetrahydrofuran at a suitable temperature in the range of about −70° C. to about 180° C. to afford compounds of Formula II. The presence of a suitable base, such as triethylamine, diisopropyl amine, potassium carbonate, sodium bicarbonate is useful in some cases to improve the yield of the reaction.

Mainly one amine of Formula VI

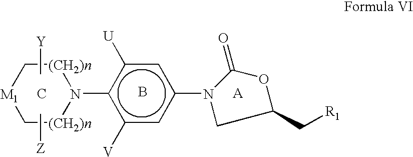

identified as a core, namely
5(S)-Isoxazol-3-yl-oxymethyl-3-[3-Fluoro-4-(piperazin-1-yl)phenyl]oxazolidin-2-one (Core 1)
   was used for analoging purposes, wherein M₁, U, V, Y, Z, R₁ and n are as defined earlier.

The key intermediate amines of Formula VI for the analogue preparation were prepared from commercially available reagents. Some amines of Formula VI are already known in the literature and are given by reference and if they have been made for the first time or by a different procedure or variation of known procedure they are described in detail in the experimental section.

The optically pure amines of Formula VI could be obtained either by one of a number of asymmetric syntheses or alternatively by resolution from a racemic mixture by selective crystallization of a salt prepared, with an appropriate optically active acid such as dibenzoyl tartrate or 10-camphorsulfonic acid, followed by treatment with base to afford the optically pure amine.

The transformations effected are described in the experimental section. In the above synthetic methods where specific acids, bases, solvents, catalysts, oxidising agents, reducing agents etc. are mentioned, it is to be understood that the other acids, bases, solvents, catalysts, oxidising agents, reducing agents etc. may be used. Similarly, the reaction temperature and duration of the reaction may be adjusted according to the desired need.

An illustrative list of particular compounds according to the invention and capable of being produced by the above mentioned schemes includes:
(S)—N-[[3-[3-Fluoro-4-[N-1-[4-{2-furyl-(5-nitro)methyl}]piperazinyl]phenyl]-2-oxo-5-oxazolidinyl]methyl]-3-(2,4-dichlorophenyl)acrylamide (Compound No. 1)
(S)—N-[[3-[3-Fluoro-4-[N-1-[4-{2-furyl-(5-nitro)methyl}]piperazinyl]phenyl]-2-oxo-5-oxazolidinyl]methyl]-3-(4-fluorophenyl)acrylamide (Compound No. 2)
(S)—N-[[3-[3-Fluoro-4-[N-1-[4-{2-furyl-(5-nitro)methyl}]piperazinyl]phenyl]-2-oxo-5-oxazolidinyl]methyl]-2-benzo(b)furanamide (Compound No. 3)
(s) —N-[3-[3-Fluoro-4-[N-1-[4-{2-furyl-(5-nitro)methyl}]piperazinyl]phenyl]-2-oxo-5-oxazolidinyl]methylamine (Compound No. 4)
(S)—N-[[3-[3-Fluoro-4-[N-1-[4-{2-furyl-(5-nitro)methyl}]piperazinyl]phenyl]-2-oxo-5-oxazolidinyl]methyl]-3-(phenyl)acrylamide (Compound No. 5)
(S)—N-[[3-[3-Fluoro-4-[N-1-[4-{2-furyl-(5-nitro)methyl}]piperazinyl]phenyl]-2-oxo-5-oxazolidinyl]methyl]-3-(1,3-benzodioxol-5-yl)acrylamide (Compound No. 6)
(S)—N-[[3-[3-Fluoro-4-[N-1-[4-{2-thienyl-(5-nitro)methyl}]piperazinyl]phenyl]-2-oxo-5-oxazolidinyl]methyl]-3-(4-fluorophenyl)acrylamide (Compound No. 7)
(S)—N-[[3-[3-Fluoro-4-[N-1-[4-{2-thienyl-(5-nitro)methyl}]piperazinyl]phenyl]-2-oxo-5-oxazolidinyl]methyl]-3-(4-nitrophenyl)acrylamide (Compound No. 8)
(S)—N-[[3-[3-Fluoro-4-[N-1-[4-{2-thienyl-(5-nitro)methyl}]piperazinyl]phenyl]-2-oxo-5-oxazolidinyl]methyl]-3-(2,4-dichlorophenyl)acrylamide (Compound No. 9)
(S)—N-[1-[[3-[3-Fluoro-4-[N-1-[4-{2-furyl-(5-nitro)methyl}]piperazinyl]phenyl]-2-oxo-5-oxazolidinyl]methyl]]-thiourea (Compound No. 10)
(S)—N-[[3-[3-Fluoro-4-[N-1-[4-{2-thienyl-(5-nitro)methyl}]piperazinyl]phenyl]-2-oxo-5-oxazolidinyl]methyl]isothiocyanate (Compound No. 11)
(S)—N-[1-[[3-[3-Fluoro-4-[N-1-[4-{2-thienyl-(5-nitro)methyl}]piperazinyl]phenyl]-2-oxo-5-oxazolidinyl]methyl]]-thiourea (Compound No. 12)
(S)—N-[[3-[3-Fluoro-4-[N-1-[4-{2-furyl-(5-nitro)methyl}]piperazinyl]phenyl]-2-oxo-5-oxazolidinyl]methyl]isothiocyanate (Compound No. 13)
5(S)-Isoxazol-3-yl-oxymethyl-3-[3-fluoro-4-[4-[(4-bromo-5-nitro-2-thienyl)methyl]piperazinyl-1-yl]phenyl]oxazolidin-2-one (Compound No. 14)
5(S)-Isoxazol-3-yl-oxymethyl-3-[3-fluoro-4-[4-[(5-nitro-2-furyl)methyl]piperazinyl-1-yl]phenyl]oxazolidin-2-one (Compound No. 15)
5(S)-Isoxazol-3-yl-oxymethyl-3-[3-fluoro-4-[4-[(5-nitro-2-thienyl)methyl]piperazinyl-1-yl]phenyl]oxazolidin-2-one (Compound No. 16)
(S)—N-[1-[[3-[3-Fluoro-4-[N-1-[4-{2-furyl-(5-nitro)methyl}]piperazinyl]phenyl]-2-oxo-5-oxazolidinyl]methyl]]3,3-dimethyl-thiourea (Compound No. 17)
(S)—N-[3-[3-Fluoro-4-[N-1-[4-{2-thienyl-(5-nitro)methyl}]piperazinyl]phenyl]-2-oxo-5-oxazolidinyl]methylamine (Compound No. 18)

Most of the compounds were characterized using NMR, IR and were purified by chromatography. Crude products were subjected to column chromatographic purification using silica gel (100-200 or 60-120 mesh) as stationary phase.

The examples mentioned below demonstrate the general synthetic procedure as well as the specific preparation for the preparation for the preferred compound. The examples are given to illustrate the details of the invention and should not be constrained to limit the scope of the present invention.

### EXAMPLE 1

### Analogues of 5(S)-Isoxazol-3-yl-oxymethyl-3-[3-Fluoro-4-piperazin-1-yl)phenyl]oxazolidin-2-one (Core I)

The heteroaromatic group with the corresponding appendage can be introduced on the nitrogen atom of ring C of compounds of Formula I by one of the methods described below:

### Method A:

### General Procedure:

The reductive alkylation of the amine intermediate of Formula VI with the corresponding heterocyclic aldehydes of the Formula VII, using known reducing agents well known to one of ordinary skill in the art, such as sodium triacetoxyborohydride or sodium cyanoborohydride gives the products of Formula II wherein W═CH₂

The following compounds were prepared using this method:

### 5(S)-Isoxazol-3-yl-oxymethyl-3-[3-fluoro-4-[4-[(4-bromo-5-nitro-2-thienyl)methyl]piperazinyl-1-yl]phenyl]oxazolidin-2-one (Compound No. 14)

To a solution of 5(S)-Isoxazol-3-yl-oxymethyl-3-[3-Fluoro-4-(piperazin-1-yl)phenyl]oxazolidin-2-one hydrochloride (0.67 mmol, prepared by procedures similar to Poster No 1825 and 1827, 40^{th} Interscience Conference on Antimicrobial Agents and Chemotherapy, Sep. 17-20, 2000, Toronto, Canada) in THF, 4-bromo-5-nitro-thiophene-2-carboxaldehyde (0.22 g, 1 mmol) and molecular sieves (0.4 g, 4 A°) were added. It was stirred for 45 min. and then sodium triacetoxyborohydride (0.21 g, 1 mmol) was added. The reaction mixture was further stirred for 17 hrs. The reaction mixture was filtered and the filtrate evaporated in vacuo. The residue obtained was taken in dichloromethane and washed with water. The organic layer was dried over anhydrous sodium sulphate and evaporated in vacuo. The residue was purified by column chromatography, eluting with 1% MeOH/CH₂Cl₂ to yield 0.097 g of the product.

¹HNMR (CDCl₃) δppm: 8.16 (d, 1H), 7.49 (dd, 1H), 7.11 (d, 1H), 6.97 (t, 1H), 6.01 (d, 1H), 5.01 (m, 1H), 4.55 (m, 2H), 4.14 (t, 1H), 3.92 (m, 1H), 3.75 (s, 2H), 3.12 (m, 4H), 2.76 (m, 4H)

Mass: M=582, M+2=582, M+Na=604

### 5(S)-Isoxazol-3-yl-oxymethyl-3-[3-fluoro-4-[4-[(5-nitro-2-furyl)methyl]piperazinyl-1-yl]phenyl]oxazolidin-2-one (Compound No. 15)

The title compound was prepared from 5(S)-Isoxazol-3-yl-oxymethyl-3-[3-Fluoro-4-(piperazin-1-yl)phenyl]oxazolidin-2-one hydrochloride and 5-nitro-2-furaldehyde using Method A and procedure similar to the preparation of compound no. 14.

m.pt: 133-135° C.

¹HNMR (CDCl₃) δppm: 8.18 (s, 1H), 7.65-6.8 (m, 5H), 6.53 (d, 1H0, 6.02 (s, 1H0, 5.02 (brs, 5H), 4.54 (m, sH), 4.2-3.9 (m, 2H0, 3.73 (m, 2H), 3.2-2.6 (m, 8H),

Mass: M=487, M+2=489, M+Na=510

### 5(S)-Isoxazol-3-yl-oxymethyl-3-[3-fluoro-4-[4-[(5-nitro-2-thienyl)methyl]piperazinyl-1-yl]phenyl]oxazolidin-2-one (Compound No. 16)

The title compound was prepared from 5(S)-Isoxazol-3-yl-oxymethyl-3-[3-Fluoro-4-(piperazin-1-yl)phenyl]oxazolidin-2-one hydrochloride and 5-nitro-thiophene-2-carboxaldehyde using Method A and procedure similar to the preparation of compound no. 14.

m.pt: 165-167° C.

¹HNMR (CDCl₃) δppm: 8.16 (d, 1H), 7.81 (d, 1H), 7.46 (dd, 1H), 7.11 (d, 1H), 6.96 (t, 1H), 6.89 (d, 1H), 6.01 (d, 1H), 5.02 (m, 1H0, 4.54 (m, 2H), 4.17 (t, 1H), 3.93 (m, 1H), 3.78 (s, 2H), 3.12 (m, 4H), 2.74 (m, 4H)

Mass: M+1=504, M+Na=526

### EXAMPLE 2

### Analogues of (S)—N-[3-[3-Fluoro-4-[N-1-[4-{2-furyl-(5-nitro)methyl}]piperazinyl]phenyl]-2-oxo-5-oxazolidinyl]methylamine (Core II)

### Preparation of (S)—N-[3-[3-Fluoro-4-[T-1-[4-{2-furyl-(5-nitro)methyl}]piperazinyl]phenyl]-2-oxo-5-oxazolidinyl]methylamine (Compound No. 4)

To (S)—N-[[3-[3-Fluoro-4-[N-1-[4-{2-furyl-(5-nitro)methyl}]piperazinyl]phenyl]-2-oxo-5-oxazolidinyl]methyl]acetamide hydrochloride (3.2 g, prepared as described in WO 02/06278) in 1N hydrochloric acid (32 mL) was heated to reflux for 4 hrs. The reaction mixture was cooled and extracted with dichloromethane. The aqueous layer was made alkaline with 1N ammonium hydroxide and extracted with dichloromethane. The organic layer was dried over anhyd.sodium sulphate and evaporated in vacuo. The crude product was crystallized with ethyl acetate/hexane to yield 1.8 g of the title compound.

### Method B:

### General Procedure:

For the preparation of compounds of Formula I wherein W is equal to C═O, the corresponding acid of Formula VII can be used and the amine of Formula VI can be acylated through activated esters in the presence of condensing agents, such as 1,3-dicyclohexylcarbodiimide (DCC) and 1-(3-dimethylaminopropyl)-3-ethylcarbodiimide hydrochloride (EDC), along with 1-hydroxybenzotriazole. Other methods of acylation can also be employed.

The following compounds were prepared using this method:

### (S)—N-[[3-[3-Fluoro-4-[N-1-[4-{2-furyl-(5-nitro)methyl}]piperazinyl]phenyl]-2-oxo-5-oxazolidinyl]methyl]-3-(2,4-dichlorophenyl)acrylamide (Compound No. 1)

To (S)—N-[3-[3-Fluoro-4-[N-1-[4-{2-furyl-(5-nitro)methyl}]piperazinyl]phenyl]-2-oxo-5-oxazolidinyl]methylamine (0.3 g, 0.71 mmol) in DMF (10 nm-L), N-methylmorpholine (0.088 g, 0.85 mmol), 1-hydroxybenzotriazole (0.11 g, 0.79 mmol) and 2,4-dichlorocinnamic acid (0.19 g, 0.85 mmol) were added at 0° C. The reaction mixture was stirred at 0° C. for 30 min. and then EDC (0.16 g, 0.85 mmol) was added. The reaction mixture was further stirred for 17 hrs. It was poured into water and extracted with ethyl acetate. The organic layer was dried over anhyd sodium sulphate and concentrated in vacuo. The residue obtained was purified by column chromatography.

¹H NMR (CDCl₃) δPPM: 7.93 (d, 1H), 7.42 (m, 3H), 7.28 (m), 7.06 (dd, 1H), 6.90 (t, 1H), 6.51 (m, 2H), 6.43 (d, 1H), 4.82 (m, 1H), 4.04 (t, 1H), 3.83 (m, 3H), 3.71 (s, 2H), 3.07 (m, 4H), 2.71 (m, 4H).

Mass: M+1=618, M+Na=640.

### (S)—N-[[3-[3-Fluoro-4-[N-1-[4-{2-furyl-(5-nitro)methyl}]piperazinyl]phenyl]-2-oxo-5-oxazolidinyl]methyl]-3-(4-fluorophenyl)acrylamide (Compound No. 2)

The title compound was prepared from (S)—N-[3-[3-Fluoro-4-[N-1-[4-{2-furyl-(5-nitro)methyl}]piperazinyl]phenyl]-2-oxo-5-oxazolidinyl]methylamine and 4-fluorocinnamic acid using Method B and procedure similar to the preparation of compound no. 1

¹H NMR (CDCl₃) δPPM: 7.60 (dd, 1H), 7.49-7.45 (m, 2H), 7.41-7.40 (m, 2H), 7.08-7.03 (m, 2H), 6.92 (t, 1H), 6.51 (d, 1H), 6.37 (d, 1H), 6.32 (d, 1H), 6.25 (br s, 1H), 4.84-4.79 (m, 1H), 4.04 (t, 1H), 3.83-3.70 (m, 5H), 3.07-3.06 (m, 4H), 2.7 (m, 4H).

Mass: M+1=568

### (S)—N-[[3-[3-Fluoro-4-[N-1-[4-{2-furyl-(5-nitro)methyl}]piperazinyl]phenyl]-2-oxo-5-oxazolidinyl]methyl]-2-benzo(b)furanamide (Compound No. 3)

The title compound was prepared from (S)—N-[3-[3-Fluoro-4-[N-1-[4-{2-furyl-(5-nitro)methyl}]piperazinyl]phenyl]-2-oxo-5-oxazolidinyl]methylamine and benzo(b)furan-2-carboxylic acid using Method B and procedure similar to the preparation of compound no. 1

¹H NMR (CDCl₃) δPPM: 7.68(d, 1H), 7.51-7.39 (m, 3H), 7.33-7.29 (m, 2H), 7.08 (d, 2H), 6.90 (t, 1H), 6.50 (d, 1H), 4.9 (m, 1H), 4.05 (t, 1H), 3.97-3.93 (m, 1H), 3.85-3.80 (m, 2H), 3.49-3.47 (m, 2H), 3.08-3.06 (m, 4H), 2.72-2.71 (m, 4H).

### (S)—N-[[3-[3-Fluoro-1-[N-1-[4-{2-furyl-(5-nitro)methyl}]piperazinyl]phenyl]-2-oxo-5-oxazolidinyl]methyl]-3-(phenyl)acrylamide (Compound No. 5)

The title compound was prepared from (S)—N-[3-[3-Fluoro-4-[N-1-[4-{2-furyl-(5-nitro)methyl}]piperazinyl]phenyl]-2-oxo-5-oxazolidinyl]methylamine and cinnamic acid using Method B and procedure similar to the preparation of compound no. 1

¹H NMR (CDCl₃) δPPM: 7.63(dd, 1H), 7.48-7.44 (m, 2H), 7.37 (s, 5H), 7.29 (m, 1H), 7.05 (d, 1H), 6.89 (t, 1H), 6.50-6.49 (d, 1H), 6.26 (m, 1H), 4.71 (m, 1H), 4.04 (t, 1H), 3.82-3.77 (m, 3H), 3.70 (m, 2H), 3.08-3.05 (m, 4H), 2.72-2.69 (m, 4H).

### (S)—N-[[3-[3-Fluoro+[N-1-[4-{2-furyl-(5-nitro)methyl}]piperazinyl]phenyl]-2-oxo-5-oxazolidinyl]methyl]-3-(1,3-benzodioxol-5-yl)acrylamide (Compound No. 6)

The title compound was prepared from (S)—N-[3-[3-Fluoro-4-[N-1-[4-{2-furyl-(5-nitro)methyl}]piperazinyl]phenyl]-2-oxo-5-oxazolidinyl]methylamine and 3-(1,3-benzodioxol-5-yl)acrylic acid using Method B and procedure similar to the preparation of compound no. 1

¹H NMR (CDCl₃) δPPM: 7.81-7.80 (m, 1H), 7.58-7.53 (dd, 1H), 7.50 (d, 1H), 7.07-7.05 (m, 2H), 6.99-6.97 (m, 2H), 6.9-6.89 (m, 1H), 6.82-6.79 (m, 1H), 6.23-6.19 (m, 1H), 6.01 (m, 2H), 4.84 (m, 1H), 4.05 (t, 1H), 3.84-3.77 (m, 5H), 3.11-3.08 (m, 4H), 2.7 (m, 4H).

### EXAMPLE 3

### Analogues of (S)—N-[3-[3-Fluoro-4-[N-1-[4-{2-thienyl-(5-nitro)methyl}]piperazinyl]phenyl]-2-oxo-5-oxazolidinyl]methylamine (Core III)

### (S)—N-[3-[3-Fluoro-4-[N-1-[4-{2-thienyl-(5-nitro)methyl}]piperazinyl]phenyl]-2-oxo-5-oxazolidinyl]methylamine (Compound No. 18)

The title compound was prepared from (S)—N-[[3-[3-Fluoro-4-[N-1-[4-{2-thienyl-(5-nitro)methyl}]piperazinyl]phenyl]-2-oxo-5-oxazolidinyl]methyl]acetamide (prepared as described in patent application WO 02/06278) and 1N HCl using the procedure similar to the preparation of compound no. 4.

### (S)—N-[[3-[3-Fluoro-4-[N-1-[4-{2-thienyl-(5-nitro)methyl}]piperazinyl]phenyl]-2-oxo-5-oxazolidinyl]methyl]-3-(4-fluorophenyl)acrylamide (Compound No. 7)

The title compound was prepared from (S)—N-[3-[3-Fluoro-4-[N-1-[4-{2-thienyl-(5-nitro)methyl}]piperazinyl]phenyl]-2-oxo-5-oxazolidinyl]methylamine and 4-fluorocinnamic acid using Method B and procedure similar to the preparation of compound no. 1

¹H NMR (CDCl₃) δPPM: 7.79 (d, 1H), 7.62-7.57 (dd, 1H), 7.49-7.41 (m, 5H), 7.08-7.03 (m, 3H), 6.91-688(m, 2H), 6.37-6.32 (dd, 1H), 6.24 (m, 1H), 4.83 (m, 1H), 4.05 (t, 1H), 3.86-3.76 (m, H), 3.08-3.07 (m, 4H), 2.72 (m, 4H).

Mass: M+1=584

### (S)—N-[[3-[3-Fluoro-4-[N-1-[4-{2-thienyl-(5-nitro)methyl}]piperazinyl]phenyl]-2-oxo-5-oxazolidinyl]methyl]-3-(4-nitrophenyl)acrylamide (Compound No. 8)

The title compound was prepared from (S)—N-[3-[3-Fluoro-4-[N-1-[4-{2-thienyl-(5-nitro)methyl}]piperazinyl]phenyl]-2-oxo-5-oxazolidinyl]methylamine and 4-nitrocinnamic acid using Method B and procedure similar to the preparation of compound no. 1.

¹H NMR (CDCl₃) δPPM: 8.21 (d, 1H), 7.80 (d, 1H), 7.69-7.60 (m, 3H), 7.48-7.43 (dd, 1H), 7.05 (d, 1H), 6.94-6.91 (m, 2H), 6.62-6.57 (m, 2H), 4.87 (m, 1H), 4.07 (t, 1H), 3.84-3.78 (m, 5H), 3.09 (m, 4H), 2.74 (m, 4H).

Mass: M+1=611

### (S)—N-[[3-[3-Fluoro-4-[N-1-[4-{2-thienyl-(5-nitro)methyl}]piperazinyl]phenyl]-2-oxo-5-oxazolidinyl]methyl]-3-(2,4-dichlorophenyl)acrylamide (Compound No. 9)

The title compound was prepared from (S)—N-[3-[3-Fluoro-4-[N-1-[4-{2-thienyl-(5-nitro)methyl}]piperazinyl]phenyl]-2-oxo-5-oxazolidinyl]methylamine and 2,4-dichlorocinnamic acid using Method B and procedure similar to the preparation of compound no. 1.

¹H NMR (CDCl₃) δPPM: 7.96-7.91 (dd, 1H), 7.51-7.42 (m, 3H), 7.26-7.21 (m, 2H), 7.07-7.04 (m, 1H), 6.93-6.88 (m, 2H), 6.58-6.56 (m, 1H), 6.47-6.42 (dd, 1H), 4.85 (m, 1H), 4.05 (t, 1H), 3.82-3.76 (m, 5H), 3.08 (m, 4H), 2.72 (m, 4H).

Mass: M+1=634.

### (S)—N-[[3-[3-Fluoro-4-[N-1-[4-{2-furyl-(5-nitro)methyl}]piperazinyl]phenyl]-2-oxo-5-oxazolidinyl]methyl]isothiocyanate (Compound No. 13)

To (S)—N-[3-[3-Fluoro-4-[N-1-[4-{2-furyl-(5-nitro)methyl}]piperazinyl]phenyl]-2-oxo-5-oxazolidinyl]methylamine (1 g, 2.38 mmol) in THF, carbon disulfide (0.36 g, 4.77 mmol) and triethylamine (0.24 g, 2.38 mmol) were added at 0° C. The reaction mixture was stirred at RT for 5 hrs. The reaction mixture was again cooled to 0° C., ethylchloroformate (0.26 g, 2.38 mmol) was added and stirred for 2 hrs. The reaction mixture was then poured into water and extracted with ethyl acetate. The organic layer was dried over anhyd.sodium sulphate and evaporated in vacuo. The residue was purified by column chromatography, eluting with 1% MeOH/CHCl₃ to yield 0.6 g of the product.

¹H NMR (CDCl₃) δPPM: 7.40 (dd, 1H), 7.29 (t, 1H), 7.12 (d, 1H), 6.94 (t, 1H), 6.51 (d, 1H), 4.82-4.79 (m, 1H), 4.14 (t, 1H), 3.99-3.97 (m, 1H), 3.87-3.81 (m, 2H), 3.71 (m, 2H), 3.12-3.09 (m, 4H), 2.74-2.71 (m, 4H).

### Method C:

The isothiocyanates of Formula XI is reacted with (un)substituted amine to get the compounds of Formula II. The reaction is carried in a suitable solvent, such as dimethylformamide, dimethylacetamide, dichloromethane or tetrahydrofuran at a suitable temperature in the range of about −70° C. to about 180° C. to afford compounds of Formula II. The presence of a suitable base such as triethylamine, diisopropyl amine, potassium carbonate, sodium bicarbonate is useful in some cases to improve the yield of the reaction.

### (S)—N-[1-[[3-[3-Fluoro-4-[N-1-[4-{2-furyl-(5-nitro)methyl}]piperazinyl]phenyl]-2-oxo-5-oxazolidinyl]methyl]]3,3-dimethyl-thiourea (Compound No. 17)

To (S)—N-[[3-[3-Fluoro-4-[N-1-[4-{2-furyl-(5-nitro)methyl}]piperazinyl]phenyl]-2-oxo-5-oxazolidinyl]methyl]isothiocyanate (0.15 g, 0.325 mmol) in methanol (10 mL), triethylamine (0.131 g, 1.3 mmol) and dimethylamine hydrochloride (0.1 g, 1.3 mmol) were added. The reaction mixture was stirred for 2 hrs at RT, filtered and washed with methanol. The filtrate was concentrated to get 0.085 g of the final product.

¹H NMR (CDCl₃) δPPM: 7.44(dd, 1H), 7.29 (d, 1H), 7.05 (d, 1H), 6.92 (t, 1H), 6.51 (d, 1H), 5.91 (t, 1H), 4.92 (m, 1H), 4.31 (m, 1H), 4.07 (m, 2H), 3.87 (m, 1H), 3.71 (s, 2H), 3.28 (s, 6H), 3.09 (m, 4H), 2.72 (m, 4H).

Mass: M+1=507, M+Na=529.

### (S)—N-[1-[[3-[3-Fluoro-4-[N-1-[4-{2-furyl-(5-nitro)methyl}]piperazinyl]phenyl]-2-oxo-5-oxazolidinyl]methyl]]-thiourea (Compound No. 10)

The title compound was prepared from (S)—N-[[3-[3-Fluoro-4-[N-1-[4-{2-furyl-(5-nitro)methyl}]piperazinyl]phenyl]-2-oxo-5-oxazolidinyl]methyl]isothiocyanate and methanolic ammonia using Method C and procedure similar to the preparation of compound no. 17.

¹H NMR (CDCl₃) δPPM: 7.93(m, 1H), 7.66-7.65 (m, 1H), 7.48 (dd, 1H), 7.17-7.03 (m, 2H), 6.77 (d, 1H), 4.82 (m, 1H), 4.08 (t, 1H), 3.92-3.88 (m, 4H), 3.79 (m, 2H) 2.99 (m, 4H), 2.61 (m, 4H).

Mass: M+1=479

### (S)—N-[[3-[3-Fluoro-4-[N-1-[4-{2-thienyl-(5-nitro)methyl}]piperazinyl]phenyl]-2-oxo-5-oxazolidinyl]methyl]isothiocyanate (Compound No. 11)

The title compound was prepared from (S)—N-[3-[3-Fluoro-4-[N-1-[4-{2-thienyl-(5-nitro)methyl}]piperazinyl]phenyl]-2-oxo-5-oxazolidinyl]methylamine using the and procedure similar to the preparation of compound no 13.

¹H NMR (CDCl₃) δPPM: 7.8 (d, 1H), 7.45-7.41 (dd, 1H), 7.10 (d, 1H), 6.98 (d, 1H), 6.95-6.88 (m, 2H), 4.81-4.79 (m, 1H), 4.14 (t, 1H), 3.96-3.76 (m, 5H), 3.11 (m, 4H), 2.73 (m, 4H).

Mass: M+1=478

### (S)—N-[1-[[3-[3-Fluoro-4-[N-1-[4-{2-thienyl-(5-nitro)methyl}]piperazinyl]phenyl]-2-oxo-5-oxazolidinyl]methyl]]-thiourea (Compound No. 12)

The title compound was prepared from (S)—N-[[3-[3-Fluoro-4-[N-1-[4-{2-thienyl-(5-nitro)methyl}]piperazinyl]phenyl]-2-oxo-5-oxazolidinyl]methyl]isothiocyanate and methanolic ammonia using Method C and procedure similar to the preparation of as compound no. 17.

¹HNMR (DMSO) δPPM: 8.03 (d, 1H), 7.91 (t, 1H), 7.51-7.46 (dd, 1H), 7.18-7.05 (m, 4H), 4.82 m, 1H), 4.11 (t, 1H0, 3.84-3.80 (m, 5H0, 3.16 (m, 4H), 2.66 (m, 4H).

Mass: M+1=495

### EXAMPLE 4

### Pharmacological Testing

The compounds of the invention display antibacterial activity when tested by the agar incorporation method. The following minimum inhibitory concentrations (μg/ml) were obtained for representative compounds of the invention which are given below in the following table.

### Guide to Table Abbreviations:

1) S. aureus ATCC 25923—Staphylococus aureus ATCC 25923
2) MRSA 15187—Methicillin Resistant Staphylococcus aureus
3) Ent. faecalis ATCC 29212—Enterococcus faecalis ATCC 29212
4) Ent. faecium 6A—Enterococcus faecium 6A Van®, Cipro®
5) Strep. pne. ATCC 6303—Streptococcus pneumoniae ATCC 6303
6) Strep. pyog. ATCC 19615—Streptococcus pyogenes
7) S. epidermidis—Staphylococcus epidermidis ATCC 12228

TABLE
In vitro (μg/ml)

| Compd. | S. aureus | MRSA | MRSA | MRSA | E. faecalis | VRE | S. pyogenes | S. pneum | S. pneum |
|---|---|---|---|---|---|---|---|---|---|
| No. | 259231 | 15187 | 562 | 33 | 29212 | 6A | 19615 | 6303 | AB34 |
| 10 | 1 | 1 | 1 | 1 | 0.5 | 1 | 0.25 | <0.06 | 0.24 |
| 12 | 1 | 0.5 | 0.5 | 1 | 1 | 0.5 | <0.25 | 0.5 | 0.5 |

The in vitro antibacterial activity of the compounds was demonstrated by the agar incorporation method (NCCLS M 7 and M 100-S8 documents). Briefly, the compounds were dissolved in dimethylsulfoxide and doubling dilution of the compounds were incorporated into Meer Hilton agar before solidification. Inoculum was prepared by suspending 4 to 5 colonies into 5 ml of normal saline solution and adjusting the turbility to 0.5 Macfarland turbidity standard tables (1.5×10⁸ CFU/ml), after appropriate dilutions, 10⁴ CFU/spot was transferred into the surface of dried plate and incubated for 18 hours (24 hours for MRSN studies). The concentration showing no growth of the inoculated culture was recorded as the MIC. Appropriate ATCC standard strains were simultaneously tested and result recorded only when the MIC's against standard antibiotics were within the acceptable range.

While the present invention has been described in terms of its specific embodiments, certain modifications and equivalents will be apparent to those skilled in the art and are intended to be included within the scope of the present invention.

1. Compounds having the structure of Formula I: 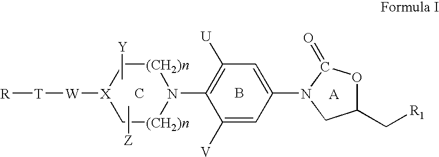
and their pharmaceutically acceptable salts, pharmaceutically acceptable solvates, esters, enantiomers, diastereomers, N-oxides, polymorphs, prodrugs or metabolites, wherein
T is a five to seven membered heterocyclic ring, substituted heterocyclic ring, aryl or substituted aryl, bound to the ring C with a linker W, and further substituted by a group represented by R, wherein R is H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, C═CH—R₅, OR₅, SR₅, —C(R₉)═C(R₉)NO₂, C₁₋₁₂ alkyl substituted with one or more of F, Cl, Br, I, OR₄, SR₄, wherein R₄ is hydrogen, alkoxy, aryl, heteroaryl, amines, substituted amines, alkene substituted with aryl, heteroaryl or halogen; R₅ is H, C₁₋₁₂ alkyl, C₃₋₁₂ cycloalkyl, C₁₋₆ alkoxy, aryl, heteroaryl or C₁₋₆ alkyl substituted with one or more of F, Cl, Br, I or OH;
R₆ and R₇ are independently H, optionally substituted C₁₋₁₂ alkyl, C₃₋₁₂ cycloalkyl, C₁₋₆ alkoxy;
R₈ and R₉ are independently H, C₁₋₆ alkyl, F, Cl, Br, I, C₁₋₁₂ alkyl substituted with one or more of F, Cl, Br, I, OR₅, SR₄, or N(R₆,R₇);
R₁₀=H, optionally substituted C₁₋₁₂ alkyl, C₃₋₁₂ cycloalkyl, C₁₋₆ alkoxy, C₁₋₆ alkyl, aryl or heteroaryl;
n is an integer in the range from 0 to 3;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl, C₁₋₄ alkylcarbonyl, C₁₋₆ alkylcarboxy, aryl or heteroaryl;
Y and Z are independently hydrogen, C₁₋₆ alkyl, C₃₋₁₂ cycloalkyl, C₀₋₃ bridging groups;
U and V are independently hydrogen, optionally substituted C₁₋₆ alkyl, F, Cl, Br, I, C₁₋₁₂ alkyl substituted with one or more of F, Cl, Br, I;
W is CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂ or SO, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl; and
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy.

2. Compounds having the structure of Formula II: 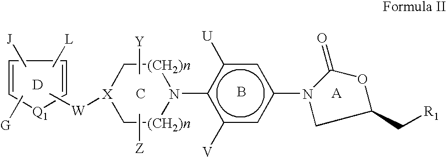
and their pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy;
U and V are independently hydrogen, optionally substituted C₁₋₆ alkyl, F, Cl, Br, I, C₁₋₁₂ alkyl substituted with one or more of F, Cl, Br, I;
Y and Z are independently hydrogen, C₁₋₆ alkyl, C₃₋₁₂ cycloalkyl, C₀₋₃ bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl carbonyl, C₁₋₄ alkylcarboxy, aryl or heteroaryl;
W is CH₂, C═O, CH₂NH, NHCH₂, CH₂NHCH₂, CH₂N(R₁₁)CH₂, CH₂N(R₁₁), CH(R₁₁), S, CH₂(C═O), NH, O, (CO)CH₂, N(R₁₁)CON(R₁₁), SO₂, SO, NR₁₁, N(R₁₁)C(═S)N(R₁₁); wherein R₁₁ is hydrogen, optionally substituted C₁₋₁₂ alkyl, C₃₋₁₂ cycloalkyl, C₁₋₆ alkoxy, C₁₋₄ alkyl carbonyl, C₁₋₆ alkylcarboxy, aryl or heteroaryl;
n is an integer in the range from 0 to 3;
Q₁ is O, S or NR₁₁, wherein R₁₁ is as defined above;
G, J, L are independently H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is as defined above; R₅ is H, C₁₋₁₂ alkyl, C₃₋₁₂ cycloalkyl, C₁₋₄ alkoxy, aryl or heteroaryl; C₁₋₆ alkyl substituted with one or more of F, Cl, Br, I or OH;
R₆ and R₇ are independently H, optionally substituted C₁₋₁₂ alkyl, C₃₋₁₂ cycloalkyl or C₁₋₄ alkoxy;
R₈ and R₉ are independently H, C₁₋₆ alkyl, F, Cl, Br, I, C₁₋₁₂ alkyl substituted with one or more of F, Cl, Br, I, OR₅, SR₄, N(R₆,R₇); and
R₁₀=H, optionally substituted C₁₋₁₂ alkyl, C₃₋₁₂ cycloalkyl, C₁₋₄ alkoxy, C₁₋₄ alkyl, aryl or heteroaryl.

3. The compound according to claim 2 wherein in Formula II, ring C is 6-8 membered in size and the ring may have either two or three carbon atoms between each nitrogen atom comprising of: 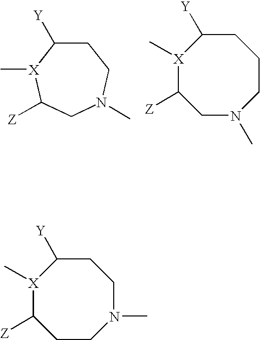
and the ring C may be bridged to form a bicyclic system as shown below:

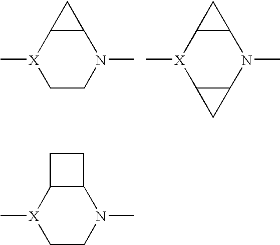

4. The compound according to claim 2 wherein in Formula II, ring C is substituted at positions Y and Z with alkyl groups, cycloalkyl groups, fluoro group, carboxylic and corresponding esters, amides, substituted alkyls or bridging alkyl groups as shown below:

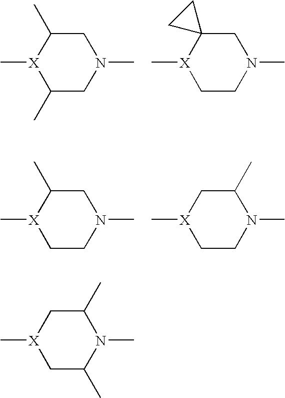

5. The compound according to claim 2 wherein in Formula II, ring C is 6 membered in size and X is —CH—(NHR₁₁), or >CCH₂NHR₁₁—, the ring C is selected from the group consisting of the following rings wherein R₁₁ is the same as defined earlier, 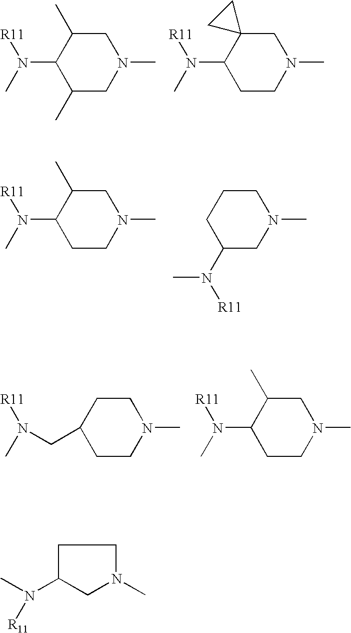
or in addition to the above, the ring C includes the following structures:

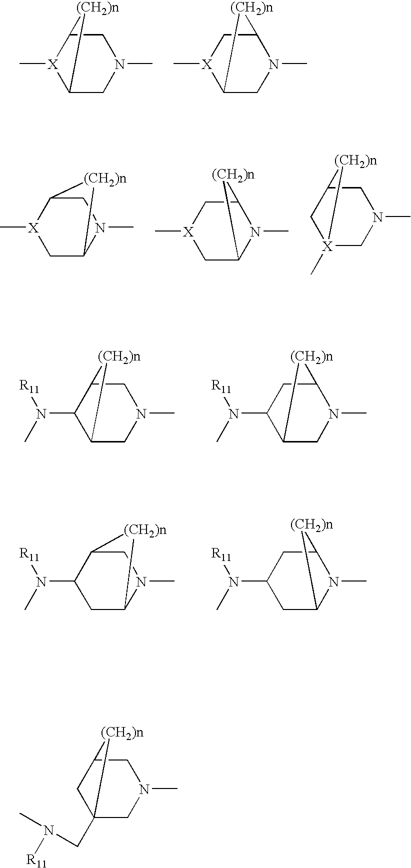

6. The compound according to claim 2 having the structure of Formula III: 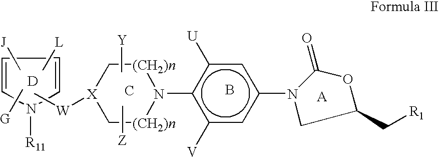
wherein U, V, Y, Z, X, W, G, J, L, R₁, R₁₁ and n are as defined earlier.

7. The compound according to claim 2 having the structure of Formula IV: 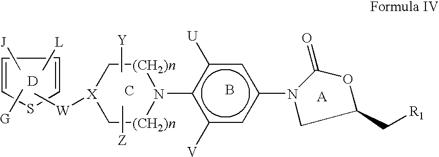
wherein U, V, Y, Z, X, W, G, J, L, R₁ and n are as defined earlier.

8. The compound according to claim 2 having the structure of Formula V: 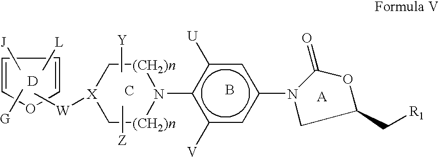
wherein U, V, X, Y, Z, W, G, J, L, R₁ and n are as defined earlier.

9. A compound selected from the group consisting of:
(S)—N-[[3-[3-Fluoro-4-[N-1-[4-{2-furyl-(5-nitro)methyl}]piperazinyl]phenyl]-2-oxo-5-oxazolidinyl]methyl]-3-(2,4-dichlorophenyl)acrylamide (Compound No. 1)
(S)—N-[[3-[3-Fluoro-4-[N-1-[4-{2-furyl-(5-nitro)methyl}]piperazinyl]phenyl]-2-oxo-5-oxazolidinyl]methyl]-3-(4-fluorophenyl)acrylamide (Compound No. 2)
(S)—N-[[3-[3-Fluoro-4-[N-1-[4-{2-furyl-(5-nitro)methyl}]piperazinyl]phenyl]-2-oxo-5-oxazolidinyl]methyl]-2-benzo(b)furanamide (Compound No. 3)
(s) —N-[3-[3-Fluoro-4-[N-1-[4-{2-furyl-(5-nitro)methyl}]piperazinyl]phenyl]-2-oxo-5-oxazolidinyl]methylamine (Compound No. 4)
(S)—N-[[3-[3-Fluoro-4-[N-1-[4-{2-furyl-(5-nitro)methyl}]piperazinyl]phenyl]-2-oxo-5-oxazolidinyl]methyl]-3-(phenyl)acrylamide (Compound No. 5)
(S)—N-[[3-[3-Fluoro-4-[N-1-[4-{2-furyl-(5-nitro)methyl}]piperazinyl]phenyl]-2-oxo-5-oxazolidinyl]methyl]-3-(1,3-benzodioxol-5-yl)acrylamide (Compound No. 6)
(S)—N-[[3-[3-Fluoro-4-[N-1-[4-{2-thienyl-(5-nitro)methyl}]piperazinyl]phenyl]-2-oxo-5-oxazolidinyl]methyl]-3-(4-fluorophenyl)acrylamide (Compound No. 7)
(S)—N-[[3-[3-Fluoro-4-[N-1-[4-{2-thienyl-(5-nitro)methyl}]piperazinyl]phenyl]-2-oxo-5-oxazolidinyl]methyl]-3-(4-nitrophenyl)acrylamide (Compound No. 8)
(S)—N-[[3-[3-Fluoro-4-[N-1-[4-{2-thienyl-(5-nitro)methyl}]piperazinyl]phenyl]-2-oxo-5-oxazolidinyl]methyl]-3-(2,4-dichlorophenyl)acrylamide (Compound No. 9)
(S)—N-[1-[[3-[3-Fluoro-4-[N-1-[4-{2-furyl-(5-nitro)methyl}]piperazinyl]phenyl]-2-oxo-5-oxazolidinyl]methyl]]-thiourea (Compound No. 10)
(S)—N-[[3-[3-Fluoro-4-[N-1-[4-{2-thienyl-(5-nitro)methyl}]piperazinyl]phenyl]-2-oxo-5-oxazolidinyl]methyl]isothiocyanate (Compound No. 11)
(S)—N-[1-[[3-[3-Fluoro-4-[N-1-[4-{2-thienyl-(5-nitro)methyl}]piperazinyl]phenyl]-2-oxo-5-oxazolidinyl]methyl]]-thiourea (Compound No. 12)
(S)—N-[[3-[3-Fluoro-4-[N-1-[4-{2-furyl-(5-nitro)methyl}]piperazinyl]phenyl]-2-oxo-5-oxazolidinyl]methyl]isothiocyanate (Compound No. 13)
5(S)-Isoxazol-3-yl-oxymethyl-3-[3-fluoro-4-[4-[(4-bromo-5-nitro-2-thienyl)methyl]piperazinyl-1-yl]phenyl]oxazolidin-2-one (Compound No. 14)
5(S)-Isoxazol-3-yl-oxymethyl-3-[3-fluoro-4-[4-[(5-nitro-2-furyl)methyl]piperazinyl-1-yl]phenyl]oxazolidin-2-one (Compound No. 15)
5(S)-Isoxazol-3-yl-oxymethyl-3-[3-fluoro-4-[4-[(5-nitro-2-thienyl)methyl]piperazinyl-1-yl]phenyl]oxazolidin-2-one (Compound No. 16)
(S)—N-[1-[[3-[3-Fluoro-4-[N-1-[4-{2-furyl-(5-nitro)methyl)}]piperazinyl]phenyl]-2-oxo-5-oxazolidinyl]methyl]]3,3-dimethyl-thiourea (Compound No. 17)
(S)—N-[3-[3-Fluoro-4-[N-1-[4-{2-thienyl-(5-nitro)methyl}]piperazinyl]phenyl]-2-oxo-5-oxazolidinyl]methylamine (Compound No. 18)

10. A pharmaceutical composition comprising the compound of claims 1, 2 or 9 and a pharmaceutical acceptable carrier.

11. A pharmaceutical composition comprising a pharmaceutically effective amount of compound according to claims 1, 2 or 9 or a physiologically acceptable acid addition salt thereof with a pharmaceutical acceptable carrier for treating microbial infections.

12. A method of treating or preventing microbial infections in a mammal comprising administering to the said mammal, the pharmaceutical composition according to claim 11.

13. The method according to claim 12 wherein the microbial infections are caused by gram-positive and gram-negative bacteria.

14. (canceled)

15. A method of treating or preventing aerobic and anaerobic bacterial infections in a mammal comprising administering to said mammal, a therapeutically effective amount of a compound having the structure of Formula I 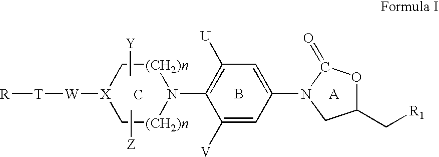
and its pharmaceutically acceptable salts, pharmaceutically acceptable solvates, esters, enantiomers, diastereomers, N-oxides, polymorphs, prodrugs or metabolites, wherein
T is a five to seven membered heterocyclic ring, substituted heterocyclic ring, aryl or substituted aryl, bound to the ring C with a linker W, and is further substituted by a group represented by R, wherein R is H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is hydrogen, alkoxy, aryl, heteroaryl, amines, substituted amines, alkene substituted with aryl, heteroaryl or halogens; R₅ is H, C₁₋₁₂ alkyl, C₃₋₁₂ cycloalkyl, C₁₋₆ alkoxy, aryl or heteroaryl; C₁₋₆ alkyl substituted with one or more of F, Cl, Br, I or OH;
R₆ and R₇ are independently H, optionally substituted C₁₋₁₂ alkyl, C₃₋₁₂ cycloalkyl, C₁₋₆ alkoxy;
R₈ and R₉ are independently H, C₁₋₆ alkyl, F, Cl, Br, I, C₁₋₁₁₂ alkyl substituted with one or more of F, Cl, Br, I, OR₅, SR₄, or N(R₆,R₇);
R₁₀=H, optionally substituted C₁₋₁₂ alkyl, C₃₋₁₂ cycloalkyl, C₁₋₆ alkoxy, C₁₋₆ alkyl, aryl or heteroaryl;
n is an integer in the range from 0 to 3;
X is H, CH, CH—S, CH—O, N, CHNR₁₁ or CCH₂NR₁₁, wherein R₁₁ is hydrogen, optionally substituted C₁₋₁₂ alkyl C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
Y and Z are independently hydrogen, C₁₋₆ alkyl, C₃₋₁₂ cycloalkyl, C₀₋₃ bridging groups;
U and V are independently hydrogen, optionally substituted C₁₋₆ alkyl, F, Cl, Br, I, C₁₋₁₂ alkyl substituted with one or more of F, Cl, Br, I;
W is CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂ or SO; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl; and
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy.

16. A method of treating or preventing aerobic and anaerobic bacterial infections in a mammal comprising administering to said mammal, a therapeutically effective amount of a compound having the structure of Formula II: 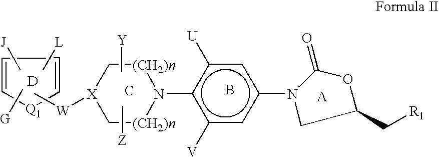
and its pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more of heteroatoms selected from O, S and N; the aryl, heteroaryl, aralkyl and aralkenyl rings may be unsubstituted or substituted with one or more of alkyl, halogen, nitro, amino or methylenedioxy;
U and V are independently hydrogen, optionally substituted C₁₋₆ alkyl, F, Cl, Br, I, C₁₋₁₂ alkyl substituted with one or more of F, Cl, Br, I;
Y and Z are independently hydrogen, C₁₋₄ alkyl, C₃₋₁₂ cycloalkyl, CO-3 bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₄ alkoxy, C₁₋₄ alkyl carbonyl, C₁₋₆ alkylcarboxy, aryl or heteroaryl;
W is CH₂, C═O, CH₂NH, NHCH₂, CH₂NHCH₂, CH₂N(R₁₁)CH₂, CH₂N(R₁₁), CH(R₁₁), S, CH₂(C═O), NH, O, (CO)CH₂, N(R₁₁)CON(R₁₁), SO₂, SO, NR₁₁, N(R₁₁)C(═S)N(R₁₁); wherein R₁₁ is hydrogen, optionally substituted C₁₋₁₂ alkyl, C₃₋₁₂ cycloalkyl, C₁₋₆ alkoxy, C₁₋₆ alkyl carbonyl, C₁₋₆ alkylcarboxy, aryl or heteroaryl;
n is an integer in the range from 0 to 3;
Q₁ is O, S or NR₁₁, wherein R₁₁ is as defined earlier;
G, J, L are independently H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₅, CHR₉, —CH═N—OR₁₀, —C═CH—R₅, OR₅, SR₅, —C(R₉)═C(R₉)NO₂, C₁₋₁₂ alkyl substituted with one or more F, Cl, Br, I, OR₄, SR₄, wherein R₄ is as defined above; R₅ is H, C₁₋₁₁₂ alkyl, C₃₋₁₂ cycloalkyl, C₁₋₆ alkoxy, aryl or heteroaryl; C₁₋₆ alkyl substituted with one or more of F, Cl, Br, I or OH;
R₆ and R₇ are independently H, optionally substituted C₁₋₁₂ alkyl, C₃₋₁₂ cycloalkyl or C₁₋₆ alkoxy;
R₈ and R₉ are independently H, C₁₋₄ alkyl, F, Cl, Br, I, C₁₋₁₂ alkyl substituted with one or more of F, Cl, Br, I, OR₅, SR₄, N(R₆,R₇); and
R₁₀=H, optionally substituted C₁₋₁₂ alkyl, C₃₋₁₂ cycloalkyl, C₁₋₆ alkoxy, C₁₋₆ alkyl, aryl or heteroaryl.

17.-19. (canceled)

20. The method according to claim 16 having the structure of Formula III, 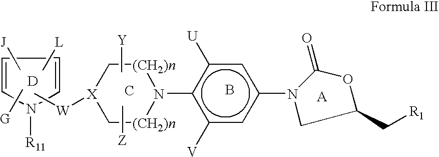
wherein U, V, Y, Z, W, X, G, J, L, R₁, R₁₁ and n are as defined earlier.

21. The method according to claim 16 having the structure of Formula IV, 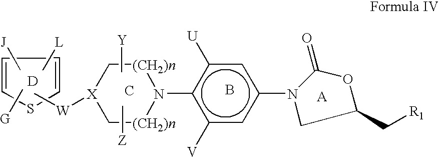
wherein U, V, Y, Z, W, X, G, J, L, R₁ and n are as defined earlier.

22. The method according to claim 16 having the structure of Formula V, 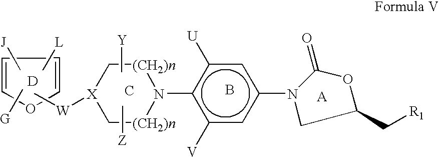
wherein U, V, X, Y, Z, W, G, J, L, R₁ and n are as defined earlier.

23. A process for preparing a compound of Formula I, 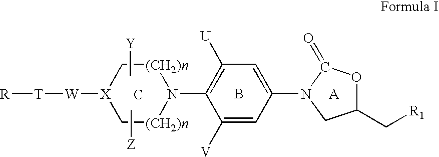
and its pharmaceutically acceptable salts, pharmaceutically acceptable solvates, esters, enantiomers, diastereomers, N-oxides, polymorphs, prodrugs or metabolites, wherein
T is a five to seven membered heterocyclic ring, substituted heterocyclic ring, aryl or substituted aryl, bound to the ring C with a linker W, and is further substituted by a group represented by R, wherein R is H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ is hydrogen, alkoxy, aryl, heteroaryl, amines, substituted amines, alkene substituted with aryl, heteroaryl or halogens; R₅ is H, C₁₋₁₂ alkyl, C₃₋₁₂ cycloalkyl, C₁₋₆ alkoxy, aryl or heteroaryl; C₁₋₆ alkyl substituted with one or more of F, Cl, Br, I or OH;
R₆ and R₇ are independently H, optionally substituted C₁₋₁₂ alkyl, C₃₋₁₂ cycloalkyl, C₁₋₆ alkoxy;
R₈ and R₉ are independently H, C₁₋₆ alkyl, F, Cl, Br, I, C₁₋₁₂ alkyl substituted with one or more of F, Cl, Br, I, OR₅, SR₄, or N(R₆,R₇);
R₁₀=H, optionally substituted C₁₋₁₂ alkyl, C₃₋₁₂ cycloalkyl, C₁₋₆ alkoxy, C₁₋₆ alkyl, aryl or heteroaryl;
n is an integer in the range from 0 to 3;
X is H, CH, CH—S, CH—O, N, CHNR₁₁ or CCH₂NR₁₁, wherein R₁₁ is hydrogen, optionally substituted C₁₋₁₂ alkyl C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl;
Y and Z are independently hydrogen, C₁₋₆ alkyl, C₃₋₁₂ cycloalkyl, C₀₋₃ bridging groups;
U and V are independently hydrogen, optionally substituted C₁₋₆ alkyl, F, Cl, Br, I, C₁₋₁₂ alkyl substituted with one or more of F, Cl, Br, I;
W is CH₂, CO, CH₂NH, —NHCH₂, —CH₂NHCH₂, —CH₂—N(R₁₁)CH₂—, CH₂(R₁₁)N—, CH(R₁₁), S, CH₂(CO), NH, O, NR₁₁, (CO)CH₂, N(R₁₁)CON(R₁₁), N(R₁₁)C(═S)N(R₁₁), SO₂ or SO; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl or heteroaryl; and
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more heteroatoms selected from O, S and N; the aryl, heteroaryl, aralkyl and aralkenyl rings may be unsubstituted or substituted with one or more of alkyl, halogen, nitro, amino or methylenedioxy;
which comprises reacting an amine of Formula VI, 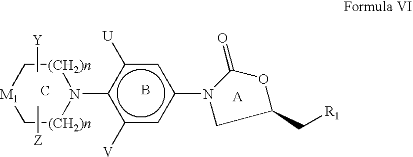
with a heteroaromatic compound of Formula R-T-W—R₁₂ wherein R, T, W, R₁, Y, Z, U, V and n are as defined earlier and M₁ is NH, NHR₁₃, CHNHR₁₃, —CHCH₂NHR₁₃, —CCH₂NHR₁₃, wherein R₁₃ is H, ethyl, methyl, isopropyl, acetyl, cyclopropyl, alkoxy or acetyl and R₁₂ is a suitable leaving group selected from the group consisting of fluoro, chloro, bromo, iodo, SCH₃, —SO₂CH₃, —SO₂CF₃, Tos, OC₆H₅, —COOH or —CHO.

24. (canceled)

25. A process for preparing a compound of Formula II, 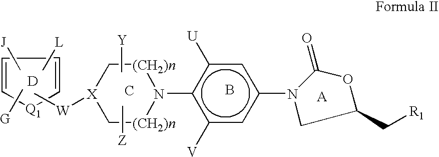
and its pharmaceutically acceptable salts, pharmaceutically acceptable solvates, esters, enantiomers, diastereomers, N-oxides, polymorphs, prodrugs or metabolites, wherein
R₁ is NHC(═O)R₂, NHC(═S)R₂, N(R₃, R₄), NR₃ or OR₃, wherein R₂, R₃, R₄ are independently hydrogen, thiocarbonyl, amines, substituted amines, aryl heteroaroyl, heterocyclic, aralkyl, aralkenyl, wherein the heteroaryl and heterocylic rings may contain one or more of heteroatoms selected from O, S and N; the aryl, heteroaryl, aralkyl and aralkenyl rings may be unsubstituted or substituted with one or more of alkyl, halogen, nitro, amino or methylenedioxy;
U and V are independently hydrogen, optionally substituted C₁₋₆ alkyl, F, Cl, Br, I, C₁₋₁₂ alkyl substituted with one or more F, Cl, Br, I;
Y and Z are independently hydrogen, C₁₋₆ alkyl, C₃₋₁₂ cycloalkyl, C₀₋₃ bridging group;
X is H, CH, CH—S, CH—O, N, CHNR₁₁ or CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl carbonyl, C₁₋₆ alkylcarboxy, aryl or heteroaryl;
W is CH₂, C═O, CH₂NH, NHCH₂, CH₂NHCH₂, CH₂N(R₁₁)CH₂, CH₂N(R₁₁), CH(R₁₁), S, CH₂(C═O), NH, O, (CO)CH₂, N(R₁₁)CON(R₁₁), SO₂, SO, NR₁₁, N(R₁₁)C(═S)N(R₁₁); wherein R₁₁ is hydrogen, optionally substituted C₁₋₁₂ alkyl, C₃₋₁₂ cycloalkyl, C₁₋₆ alkoxy, C₁₋₆ alkyl carbonyl, C₁₋₆ alkylcarboxy, aryl or heteroaryl;
n is an integer in the range from 0 to 3;
Q₁ is O, S or NR₁₁, wherein R₁₁ is as defined earlier;
G, J, L are independently H, C₁₋₆ alkyl, F, Cl, Br, I, —CN, COR₅, COOR₅, N(R₆,R₇), NHCOC(R₈, R₉, R₁₀), CON(R₆, R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more F, Cl, Br, I, OR₄, SR₄, wherein R₄ is as defined above; R₅ is H, C₁₋₁₂ alkyl, C₃₋₁₂ cycloalkyl, C₁₋₆ alkoxy, aryl or heteroaryl; C₁₋₆ alkyl substituted with one or more of F, Cl, Br, I or OH;
R₆ and R₇ are independently H, optionally substituted C₁₋₁₂ alkyl, C₃₋₁₂ cycloalkyl or C₁₋₆ alkoxy;
R₈ and R₉ are independently H, C₀₋₆ alkyl, F, Cl, Br, I, C₁₋₁₂ alkyl substituted with one or more of F, Cl, Br, I, OR₅, SR₄, N(R₆,R₇); and
R₁₀=H, optionally substituted C₁₋₁₂ alkyl, C₃₋₁₂ cycloalkyl, C₁₋₆ alkoxy, C₁₋₆ alkyl, aryl or heteroaryl;
comprising reacting a compound of Formula VI, 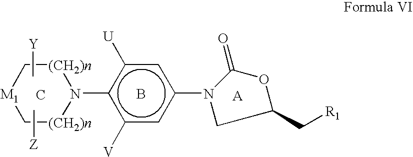
with a heteroaromatic compound of Formula VII, 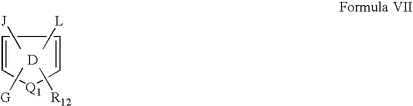
wherein R₁, U, V, Y, Z, G, J, L and Q₁ are as defined earlier and M₁ is NH, NHR₁₃, CHNHR₁₃, —CHCH₂NHR₁₃, —CCH₂NHR₁₃, wherein R₁₃ is H, ethyl, methyl, isopropyl, acetyl, cyclopropyl, alkoxy or acetyl and R₁₂ is a suitable leaving group selected from the group consisting of fluoro, chloro, bromo, iodo, SCH₃, —SO₂CH₃, —SO₂CF₃, Tos, OC₆H₅, —COOH or —CHO.

26.-41. (canceled)
